(19) United States
(12) Patent Application Publication
(10) Pub. No.: US 2025/0349422 A1
(43) Pub. Date: Nov. 13, 2025

(54) SYSTEMS AND METHODS FOR MULTILABEL TEXT CLASSIFICATION FOR AUTOMATIC LABELING OF PATIENT SELF-REPORTS

(71) Applicant: Modality.AI, Inc., San Francisco, CA (US)

(72) Inventors: Lakshmi Arbatti, Oakville (CA); Abhishek Hosamath, Brantford (CA); Vikram Ramanarayanan, San Francisco, CA (US); Ira Shoulson, Longboat Key, FL (US)

(21) Appl. No.: 18/658,198

(22) Filed: May 8, 2024

(51) Int. Cl. G16H 50/20; G06F 40/242

(52) U.S. Cl. CPC G16H 50/20; G06F 40/242

(57) ABSTRACT

Systems and methods in which a system can identify or predict one or more of a symptom and a domain from a patient's raw text query. The systems of the inventive subject matter can, based on receiving verbatims and a symptom definition table, generate a linguistic dictionary and then grow the amount of verbatims available. The verbatims are validated and used to train a model. The model is capable of predicting on or more symptoms in clinical verbiage based on a raw-text query.

### FIELD OF THE INVENTION

The field of the invention is improved computer-based patient dialog systems and methods.

### BACKGROUND

The background description includes information that may be useful in understanding the present invention. It is not an admission that any of the information provided herein is prior art or relevant to the presently claimed invention, or that any publication specifically or implicitly referenced is prior art.

Healthcare and clinical research is an information intensive industry (Wilcox and Hripcsak, 2003). The advent of Electronic Health Records (EHRs) and the availability of large amounts of clinical notes has piqued the interest of many researchers and advanced the field of Natural Language Processing (NLP). In order to automate and properly analyze and process available information, data needs to be extracted from such large corpora and arranged in a structured form understandable to computers. Classification of such information into structured reports and labels is one of the most common approaches to medical text analytics. Several pre-trained language models have been built, trained on these clinical notes and on several million other data points. These NLP algorithms have historically been used to perform such classification. One such example is the classification of free-text triage of chief complaints in pre-determined syndromic categories (Chapman et al., 2005). However, clinically-relevant classification requires expert knowledge in order to extract domain specific information and descriptors from within free text. There is no one size fits all or off the shelf solution to text analytics.

The voice of the patient has been accorded increasing research and regulatory attention, largely catalyzed by disease-focused advocacy organizations, enactment of the Twenty-First Century Cures Act, and the FDA Patient-Focused Drug Development (PFDD) initiative. What patients report about their illness is of critical importance, but has traditionally been captured using categorical scales that are rated by clinicians in research settings. Obtaining patient verbatim reports directly has not been considered feasible because of wide inter-patient variability and lack of quantification methods. However, when a patient is asked especially in a confidential online setting about what bothers them most about their disease, the responses elicited are far more nuanced and insightful compared to a face-to-face interaction with their clinical specialist that typically averages about 27 seconds and may be biased towards clinician expectations. The advent of online research platforms and maturation of medical informatics have enhanced the systematic capture and analysis of what patients experience or feel.

There are several use cases of MLTC such as genre detection (Hasan et al., 2021), topic modelling (Nawab et al., 2020) (Karvelis et al., 2018), plain medical text mining within electronic health records (EHR) (Zhang et al., 2018). Deep learning vector embedding algorithms such as Doc2Vec (Karvelis et al., 2018), Universal Sentence Encoder (Cer et al., 2018) and so on are powerful tools that can detect document similarities in a large vector space. However, they stop short in that the resulting document similarities need to be manually evaluated in order to glean additional insights from category clusters. FasTag approach to automated annotation of clinical records to match ICD-9 and ICD-10 codes for billing has yielded reasonable accuracy for veterinary data (91%) however results have been lower (71%) for human records (Venkataraman et al., 2020). Using pre-trained models such as BERT (Devlin et al., 2019) results in label classifications that are more generic since they cater to multiple input data types such as HER data or clinical notes (Turner et al., 2022). Moreover, categorizing verbatims into specific clinical symptom categories is challenging as they can be very nuanced such that different people could effectively report the same symptom in different ways. Besides, using generic pre-trained models require significant data resources and computational capabilities in the training phase (Pranji'c et al., 2020).

In the past, traditional rules-based techniques have yielded the best performance when it comes to domain heavy classification problem. A rule-based dictionary structure is simple to use and easy to implement, however they do not perform well when unknown entities are encountered and tend to result in low recall since the rules cater to very specific data sets (Houssein et al., 2021). Unfortunately, there are no tools that both capture and automatically label patient report of problems according to different categories of symptoms in a clinically meaningful manner. Hence, it is imperative that process methodology (including a pre-trained model) be created that can help classify such problem reports when applied in different disease and research settings.

Thus, there is still a need for a system that can understand a patient's plain-language input and queries to assist them in their treatment.

### SUMMARY OF THE INVENTION

The inventive subject matter provides apparatus, systems and methods in which a system can identify or predict one or more of a symptom and a domain from a patient's raw text query.

The systems and methods of the inventive subject matter includes one or more computing devices that are programmed to receive a plurality of verbatims. The verbatims can be provided from a database or other location. The computing device(s) obtain a curated symptom definition table and use it along with a known sentence structure to create a linguistic dictionary.

The computing device(s) then generates additional verbatims from the original received verbatims, and validates the additional verbatims. The total verbatim set (the original plus the additional verbatims) are then used by the computing device(s) to train a model. The trained model can then predict one or more of a symptom and a domain based on a raw-text input query.

In embodiments of the inventive subject matter, the computing device(s) can generate the linguistic dictionary by extracting parts of speech received by the computing device, training a model for synonym detection based on clinical trial and pubmed data, perform UMLS-controlled identifier extraction to obtain a plurality of words and phrases associated with a specific symptom and then extract at least one verbatim based on the obtained plurality of words and phrases.

In embodiments of the inventive subject matter, the symptom definition table can include a plurality of symptoms and, for each symptom: a domain to which the symptom belongs, at least one symptom inclusion, at least one symptom exclusion, and at least one sample phrase associated with the symptom.

In embodiments of the inventive subject matter, the computing device(s) can annotate the verbatims. In these embodiments, the annotated verbatims can include a domain to which the symptom belongs, a symptom name, a serial number, and at least one term associated with the symptom.

In embodiments of the inventive subject matter, the computer device(s) can annotate the verbatims by generating rules for each symptom based on one or more of a symptom inclusion and exclusion criteria, an obtained annotation and term or phrase, at least one closely-related term derived via algorithm, and ICD-10 codes.

Various objects, features, aspects and advantages of the inventive subject matter will become more apparent from the following detailed description of preferred embodiments, along with the accompanying drawing figures in which like numerals represent like components.

All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

The following description includes information that may be useful in understanding the present invention. It is not an admission that any of the information provided herein is prior art or relevant to the presently claimed invention, or that any publication specifically or implicitly referenced is prior art.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

### BRIEF DESCRIPTION OF THE DRAWING

FIG. 1 is a diagrammatic overview of the components of a system according to embodiments of the inventive subject matter.

FIG. 2 is a flowchart of the symptom definition phase, according to embodiments of the inventive subject matter.

FIG. 3 is a flowchart of the processes executed by the system to create a model and then use the model to match query language with symptoms and/or domains.

FIG. 4 shows a flowchart of the process of step 303 in greater detail, according to embodiments of the inventive subject matter.

FIGS. 5A-5C show an example of a symptom definition table, according to embodiments of the inventive subject matter.

FIG. 6 shows an example of an annotated verbatim, according to embodiments of the inventive subject matter.

FIG. 7 shows a table of performance between the baseline and annotated machine-learning models, according to an example of the inventive subject matter.

FIG. 8 shows an example of UMLS-CUI extraction, according to embodiments of the inventive subject matter.

FIG. 9 is a flowchart of the process executed by the computing device 110 at step 307, according to embodiments of the inventive subject matter.

FIG. 10 is an example of the interface used by a client on the client computing device to enter their plain-text input, according to embodiments of the inventive subject matter.

### DETAILED DESCRIPTION

Throughout the following discussion, numerous references will be made regarding servers, services, interfaces, engines, modules, clients, peers, portals, platforms, or other systems formed from computing devices. It should be appreciated that the use of such terms, is deemed to represent one or more computing devices having at least one processor (e.g., ASIC, FPGA, DSP, x86, ARM, ColdFire, GPU, multi-core processors, etc.) programmed to execute software instructions stored on a computer readable tangible, non-transitory medium (e.g., hard drive, solid state drive, RAM, flash, ROM, etc.). For example, a server can include one or more computers operating as a web server, database server, or other type of computer server in a manner to fulfill described roles, responsibilities, or functions. One should further appreciate the disclosed computer-based algorithms, processes, methods, or other types of instruction sets can be embodied as a computer program product comprising a non-transitory, tangible computer readable media storing the instructions that cause a processor to execute the disclosed steps. The various servers, systems, databases, or interfaces can exchange data using standardized protocols or algorithms, possibly based on HTTP, HTTPS, AES, public-private key exchanges, web service APIs, known financial transaction protocols, or other electronic information exchanging methods. Data exchanges can be conducted over a packet-switched network, the Internet, LAN, WAN, VPN, or other type of packet switched network.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

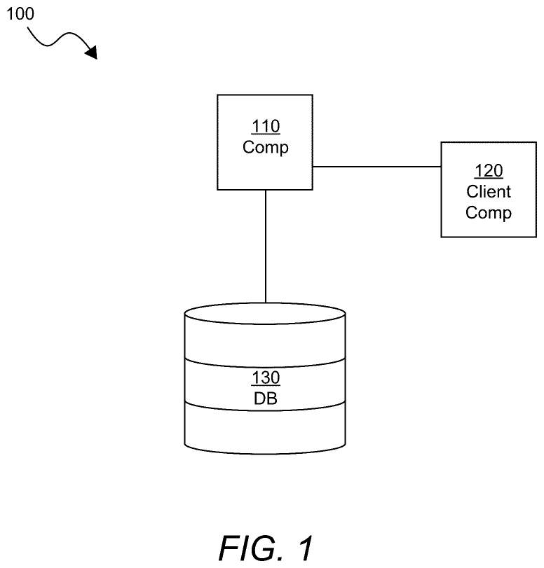

FIG. 1 is a diagrammatic overview of a system 100, according to embodiments of the inventive subject matter.

The system 100 of FIG. 1 includes a computing device 110. The computing device 110 can include one or more processors and one or more non-transitory computer-readable storage media (e.g., RAM, ROM etc) that can store code that the computing device 110 executes to carry out the processes discussed herein. The computing device 110 can connect via data exchange networks (e.g., the Internet) with other computing devices. On such example is patient computing device 120, where a patient can interact with the system. The computing device 110 can also be communicatively connected with databases such as database 130, which can store data such as the verbatims, the model data, or other data associated with the inventive subject matter.

The computing device 110 is represented as a single computing device in FIG. 1. However, it is contemplated that the computing device 110 can be more than one computing device that distributes the processes discussed herein among the more than one computing device.

The approach of the inventive subject matter involves three general steps:
1) An initial analysis of verbatims to provide the curation team with the knowledge they need to define disease-specific symptoms and domains. This also helps define a rules-based process to build a linguistic dictionary comprising of synonyms and similar terms and phrases;
2) The system applies the rules and generated linguistic dictionary to scale the data across the entire cohort; and
3) A deep learning model is trained to perform multi-label text classification (“MLTC”).

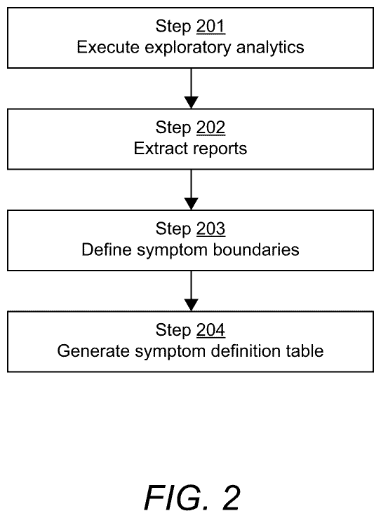

FIG. 2 is a flowchart of the symptom definition phase, according to embodiments of the inventive subject matter.

At step 201, the computing device 110 executes exploratory analytics to determine words and associated symptoms.

At step 202, the computing device 110 extracts reports based on exploratory analysis for clinical analysis.

At step 203, the curation team defines symptom boundaries. The symptom boundaries can include inclusion/exclusion criteria and common terms and phrase.

At step 204, the computing device 110 generates a symptom definition table based on the defined symptom boundaries.

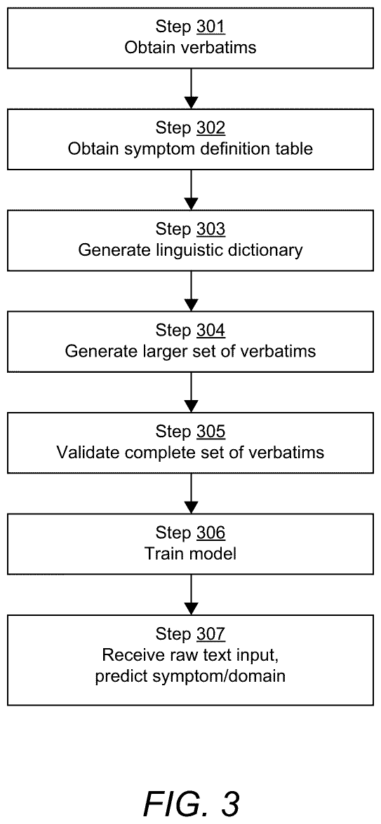

FIG. 3 is a flowchart of the processes executed by system 100 to create a model and then use the model to match query language with symptoms and/or domains, according to embodiments of the inventive subject matter.

At step 301, the computing device 110 obtains a plurality of verbatims. A verbatim can be considered to be data item containing a concatenated problem and consequence as verbally reported by a patient. For example, a problem can be an answer provided by a patient to a question about what bothers the patient about their disease, such as “What is the most bothersome problem for you due to your Parkinson's disease”. The consequence can be considered to be an answer given by a patient to a question regarding how the disease affects their daily functioning, for example “In what way does this problem bother you (by affecting your everyday functioning or ability to accomplish what needs to be done)?” The consequence is the section in parenthesis in this example. The verbatim can thus be considered to comprise a data item including the merged responses to these two types of questions.

Verbatims can be a priori gathered and stored in a database, which can then be accessed by the computing device 110.

At step 302, the computing device 110 obtains a curated symptom definition table. The curated table can be generated according to the process of FIG. 2, or obtained from a separate source.

In embodiments of the inventive subject matter, the symptom definition table can include a plurality of symptoms and, for each symptom, include a domain to which the symptom belongs, at least one symptom inclusion, at least one symptom exclusion, and at least one sample phrase associated with a symptom.

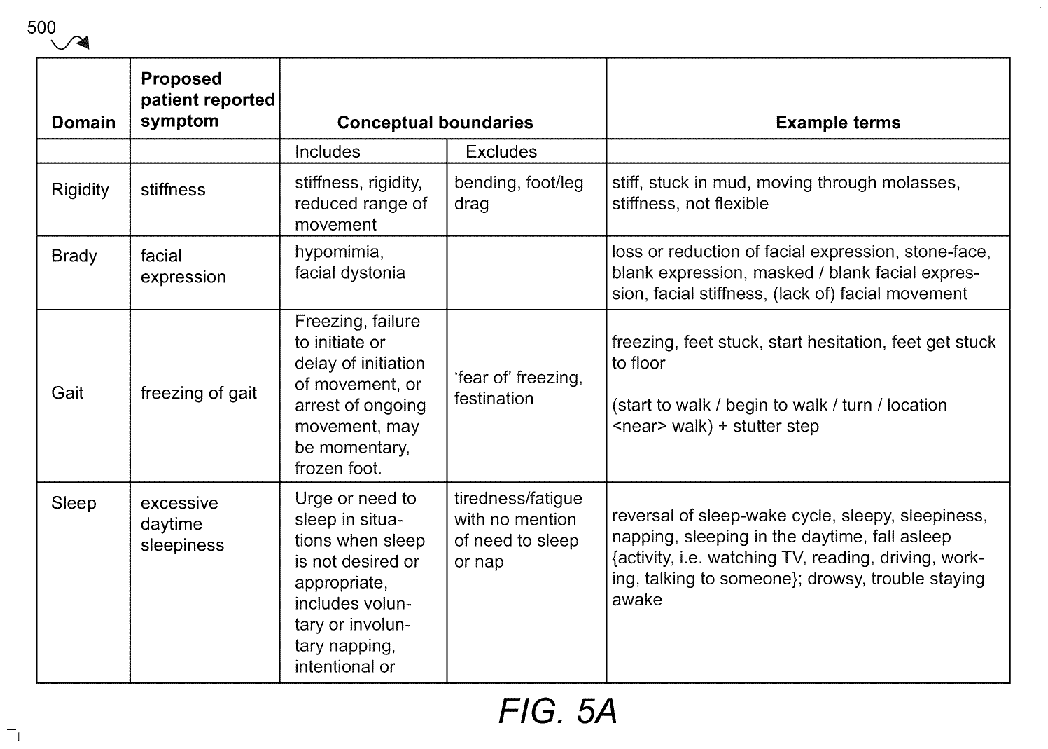

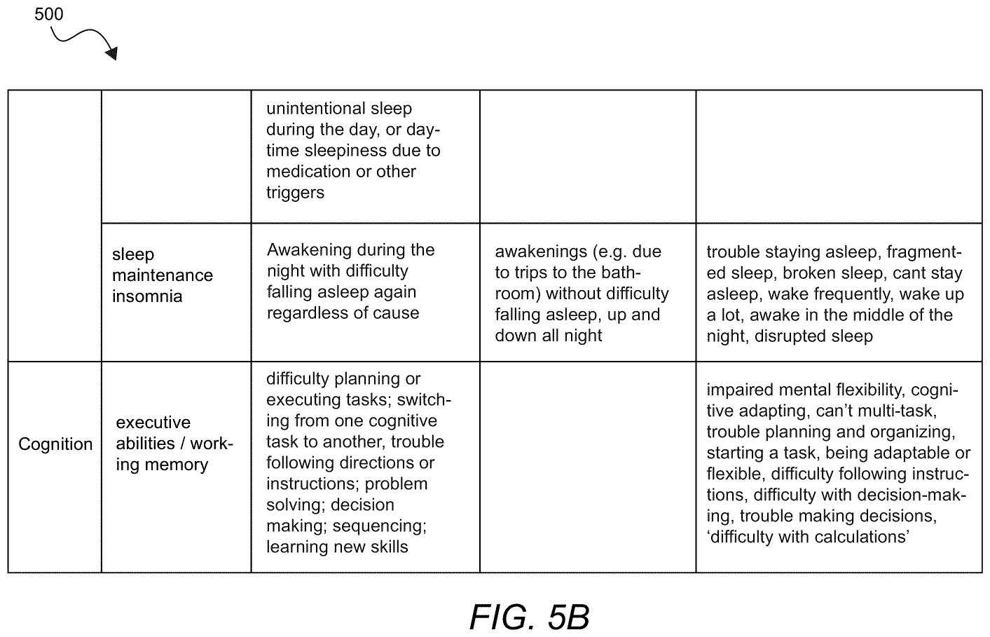

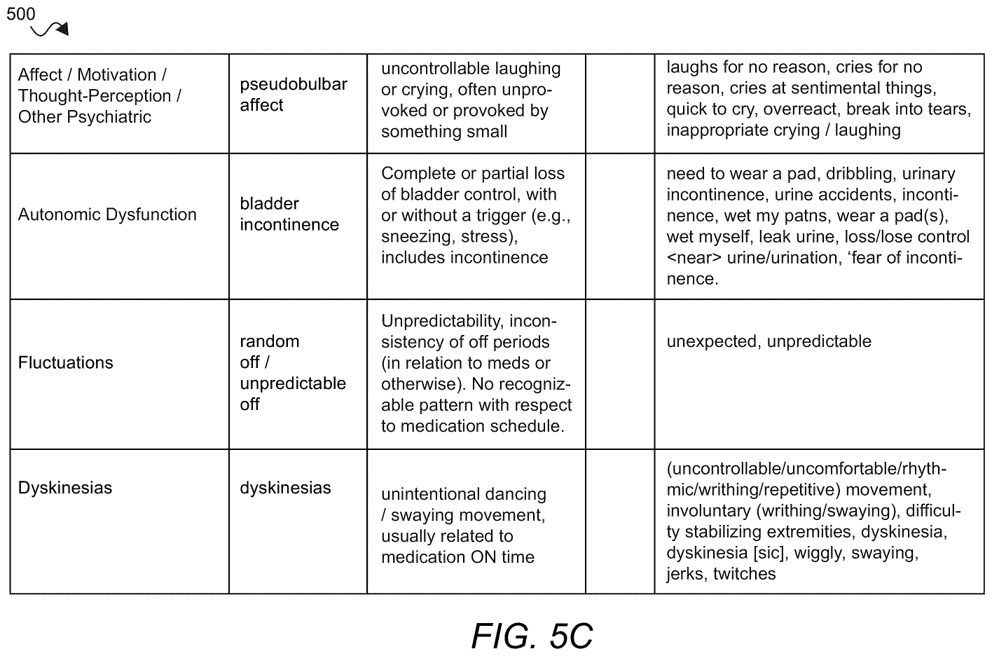

FIGS. 5A-5C is an example of a symptom definition table 500, according to embodiments of the inventive subject matter. The table 500 includes a domain column 510, a symptom column 520, a symptom inclusion column 530, a symptom exclusion column 540 and a column 550 with example terms and phrases associated with the symptom. Some domains can have a plurality of associated symptoms, as is illustrated by the “Sleep” domain in table 500.

At step 303, the computing device 110 generates a linguistic dictionary based on a known sentence structure and the curated definition table.

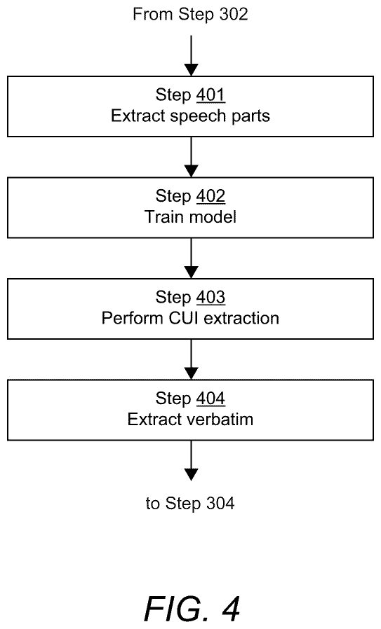

FIG. 4 shows a flowchart of the process of step 303 in greater detail, by which the computing device 110 generates the linguistic dictionary, according embodiments of the inventive subject matter.

At step 401, the computing device 110 extracts parts from received speech. The computing device 110 uses parts of speech (e.g., nouns, adjectives and verbs) from the verbatims to generate a visualization of the various aspects of symptom reporting, at which point the computing device defines the domains and symptoms.

At step 402, the computing device 110 trains a model for synonym detection based on clinical trial and published medical (“pubmed”) data. In this example, pubmed data is considered to be data from published materials from the PubMed database run by the National Library of Medicine. However, other sources of data are contemplated in addition to or instead of PubMed.

In embodiments, the computing device 110 employs a word2vec model trained on clinical trials and pubmed data for synonym detection. This enables the computing device 110 to associate conditions or symptoms as per their clinical or scientific names with the associated terms or synonyms typically used by patients when reporting. For example, when the word2vec model was queried to provide 4 terms that had the highest probability of being similar to “dystonia” a condition mentioned by patients, the model was able to correctly identify certain synonyms such as cramping, calf, ankle as other terms commonly used in a context similar to those reporting dystonia as their bothersome problem.

The process of step 402 can include the following substeps, according to embodiments of the inventive subject matter:

First, the computing device 110 downloads pubmed data. As mentioned above, pubmed data can generally refer to published medical data from sources as PubMed, wiki, ClinicalTrials, etc.

Second, the computing device 110 breaks down the information into sentences. This can be performed, for example, by using Hadoop Mapreduce.

Third, the computing device 110 tokenizes the verbatims. As is known in Natural Language Processing (“NLP”) and machine learning, tokenization refers to the process of converting a sequence of text into smaller parts, known as tokens. The tokens can be as small as a characters or as long as words.

Fourth, the computing device 110 then builds a customized vocabulary on the tokens.

Fifth, the computing device 110 generates word embeddings and trains the model.

Sixth, the computing device 110 then saves the keyed vectors.

Seventh, the computing device 110 feeds the words/phrases to the symptom table.

Eight, the computing device 110 records the synonyms.

At step 403, the computing device 110 performs UMLS-controlled unique identifier extraction to obtain a plurality of words and phrases. The execution of the UMLS-controlled extraction develops a comprehensive table of all possible terms and phrases that patients would typically use to describe a specific symptom thereby resulting in the linguistic dictionary that could then be passed into an expert system to extract appropriate verbatims to scale.

The process of step 403 can include the following substeps, according to embodiments of the inventive subject matter.

First, the computing device 110 feeds the words/phrases from the symptom table to the UMLS via an API.

Second, the computing device 110 extracts Concept Unique Identifiers (“CUIs”).

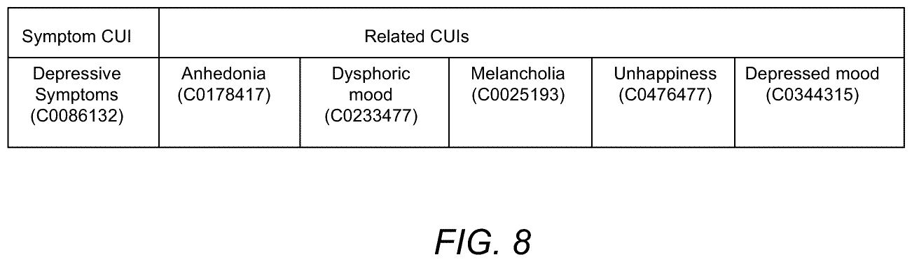

FIG. 8 shows an example of UMLS-CUI extraction 800, according to embodiments of the inventive subject matter. Taking the example verbatim: “depression, very unhappy about movement problems, fear of the future (I can't do the things I used to easily do)”. In the example of FIG. 8, it can be seen that the related terms such as “Unhappiness (C0476477)” were used to build the linguistic dictionary for Depressive Symptoms (C0086132).

Third, the computing device 110 obtains synonyms of the extracted CUIs within the UMLS system and records the synonyms.

The computing device 110 can also feed the words/phrases from the symptom table into a thesaurus as well to obtain additional synonyms, and record those.

At step 404, the computing device 110 extracts at least one verbatim from the plurality of words and phrases.

The output of step 404 then is a generated linguistic dictionary. The linguistic dictionary can be considered to be a comprehensive list of synonyms of symptoms.

The process then continues to step 304. At step 304, the computing device 110 generates a larger set of verbatims from the extracted verbatims of step 404.

The process of step 304 can include the following substeps:

The computing device 110 builds a graph database data for scalability and logical retrieval of data. The graph database can comprise patient data, visit data, and other data that can be organized into nodes containing the patient, visit and verbatim data.

The computing device then traverses through the rule-based system and, for each rule corresponding to a symptom:
A) Queries nodes using the mentioned rules and corresponding search techniques/fulltext search capabilities such as single-term query, phrase query, wildcard query, range query, regex query, fuzzy query, etc.; and
B) labels the verbatims as matched to the relevant domain-symptom categories.

The computing device 110 repeats the process until all the verbatims have been labeled, and stores the verbatims in a separate data file. The output of this process is a multi-labeled verbatim dataset.

In an example of the inventive subject matter executing the processes of step 304, NEO4j's full-text search engine powered by Apache Lucene was used along with custom search rules defined in the linguistic dictionary building steps to scale the annotation from the curated 2,341 verbatims to the entire dataset of over 170,141 verbatims. It is understood that the number of curated verbatims and scaled verbatims can vary based on a variety of factors.

The entire dataset comprising verbatims and all other participant and visit-related information was organized in Neo4j's graph schema that uses nodes and relationships to store data. Indexes were set on unique identifying information such as ParticipantID for high database operational performance.

Study level information such as demographics was stored in patient nodes, under which multiple linked visit nodes were created to store visit-level information such as visit number and verbatim reports for each visit. Extension nodes for each visit node were further created and indexed to store the concatenated verbatim problem and consequence reports for high-speed querying capabilities. All nodes were linked to each other through relationships.

Methodical rules were developed for the annotation of symptoms labels using full-text search capabilities such as single-term query, phrase query, wildcard query, range query, regex query, fuzzy query, etc., These rules were created for each symptom and stored in the linguistic dictionary based on the following:
Symptom Inclusion and Exclusion criteria provided by curation team
Annotation and terms/phrases provided by curators
Closely related terms derived from the algorithm defined while building the linguistic dictionary
ICD-10 codes.

A Python script can be used to connect to the Neo4j database for annotation and extraction of the verbatim data. Queries were used in conjunction with the rules to loop through all verbatims and annotate them. The resulting data was further reshaped with unclassified and blank rows removed using Pandas to obtain a comprehensible analytical dataset comprising verbatims and their associated symptoms.

In embodiments of the inventive subject matter, the symptoms in the validation dataset were enhanced with “negatives”, which were non-symptoms but closely related to the symptoms being validated (e.g., the sleep symptom enriched with verbatims predicted to report fatigue). The following are example verbatims of closely related symptoms:
Internal tremor: “slowness in doing things, internal quivering. (it's not something I had 315 to deal with before my disease)”
Anxiety: “Anxiety-generalized. (Makes it hard to speak publicly and voice quivers”

The results of the validation were then compared to the machine classification, after which the constructed rules were further optimized/fine-tuned for symptoms with lower accuracy. The optimized model was able to annotate verbatims with an accuracy of 96-100% for various symptoms from 1555 verbatims as manually validated by curators.

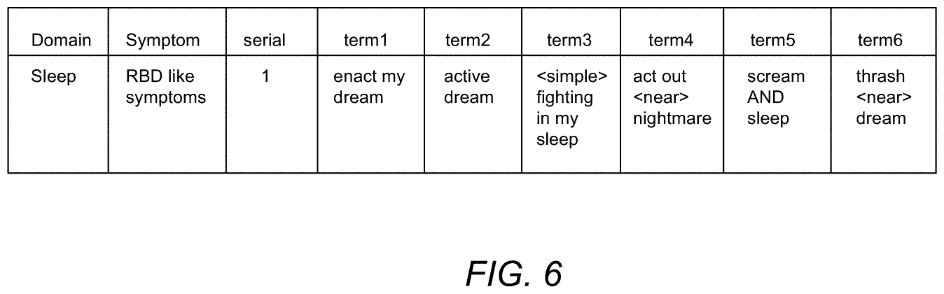

FIG. 6 is an example of an annotated verbatim 600. As can be seen in FIG. 6, the annotated verbatim 600 includes a domain to which the symptom belongs, a symptom name, a serial number, and at least one term associated with the symptom.

The larger set of verbatims is validated by the computing device 110 at step 305. In one example, approximately 1% of the analytical dataset was randomly sampled for each symptom, for validation by human curators. To further challenge the capability of the rules and machine classification, symptoms in the validation dataset were enhanced with “negatives” which were non-symptoms but closely related to the symptom being validated (e.g., the sleep symptom samples were enriched with verbatims predicted to report fatigue).

At step 306, the computing device 110 uses the validated verbatim set to train a model. The process executed by the computing device 110 at step 306 can be broken down into the following substeps:

First, the computing device 110 splits the data in the multi-labeled verbatim datasets into training, testing and validation datasets.

The computing device 110 then encodes and vectorizes the classified labeled verbatim data so it can be fed to a deep-learning neural network model (this can be considered data pre-processing steps).

The computing device 110 then proceeds to build a deep learning model for the multi-label text classification and trains the model on the training dataset.

The computing device 110 continues training the model and evaluating the model output against the validation dataset until certain pre-defined metrics (e.g., accuracy, F1-score, etc.) are reached.

The model is then frozen by the computing device 110 and the performance is evaluated using the testing dataset.

The computing device 110 then creates an inference model by combining the encoding and vectorizing layers and the saved model layers to serve as the general modal that can read raw text inputs and make predictions. Thus, the output of the process is a deep neural network inference language model.

The following is an example of the processes executed by the computing device 110 to train a model, according to embodiments of the inventive subject matter. Of the total 170,141 available verbatim samples generated in the above-described steps, 2,341 were annotated by humans. About 445 of uniformly distributed samples from this set was then set aside as the held-out test set for model evaluation. The remaining 1896 samples, constituted the “baseline” training set. The “baseline model” was trained using this set. The second evaluation set was created from the remaining 167,800 samples that were annotated by the process described herein. The trained model on this set constituted the “machine annotated model”. Both models were trained using the same Tensorflow Keras10 deep learning model architecture.

When building the machine annotated model, of the unique label classifications of 10,519 unique multi label combinations, rare label classifications i.e., label combinations with frequency of 1 were eliminated from the dataset to remove class imbalance, resulting 159,115 verbatims available for model training.

A train-test split of 90-10 was used. The test set was further split into test and validation sets at 50-50 ratio. Sklearn's train-test-split was used to achieve this task resulting in 143,203 reports in the training set, 7956 test and 7956 validation samples. The data was pre-processed by multi-hot encoding using Keras' StringLookup function and transformed into vectors using the Keras TextVectorization function, which transformed data into bi-grams first and then represented them using TD-IDF (term frequency-inverse document frequency).

The deep learning model architecture consisted of two fully connected hidden layers with 512 and 256 neurons and one output layer was then constructed. Among the several combinations of activation functions that were run, a combination of ReLU (Rectified Linear Unit) for the hidden layers and sigmoid units for the output layer yielded the best results. Given the complexity of the label combinations in the training data, ReLU was best suited to be used for the model hidden layers given its simplicity and its ability to avoid the vanishing gradient problem encountered with sigmoid or tanh functions. The sigmoid function for output layer was chosen since the predicted output was binary for each label in the multi-label classification model. The model was then compiled with a binary cross entropy loss function, given that the prediction was a 0 or 1 for each class the verbatim was to be classified into. An optimal run of 50 epochs yielded the best combination of accuracy, F1 score, precision and recall based on empirical experimentation.

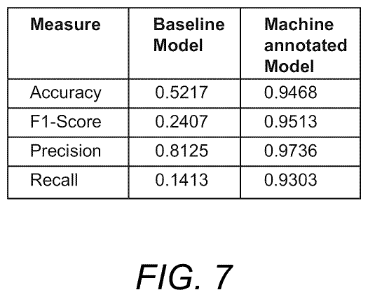

The machine-annotated model outperformed the baseline model on the held-out test on every metric as seen in FIG. 7. The accuracy improved by 42% and recall improved by 79% indicating the machine annotated model's efficiency in identifying specific multi-label combinations.

The model(s) generated can be stored by the computing device 110 and accessed in response to a query. At step 307, the computing device 110 receives a raw-text input query and uses the model to predict at least one symptom, at least one domain, and/or both in response to the raw-text query.

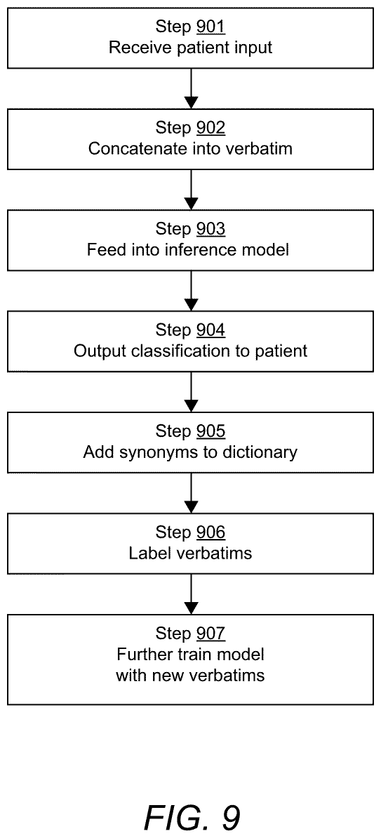

FIG. 9 is a flowchart of the process executed by the computing device 110 at step 307.

At step 901, a user enters answers questions about their disease. The questions are generally two-fold: (1) What is the most bothersome problem for you due to your disease/condition? (2) In what way does this problem bother you by affecting your everyday functioning or ability to accomplish what needs to be done?

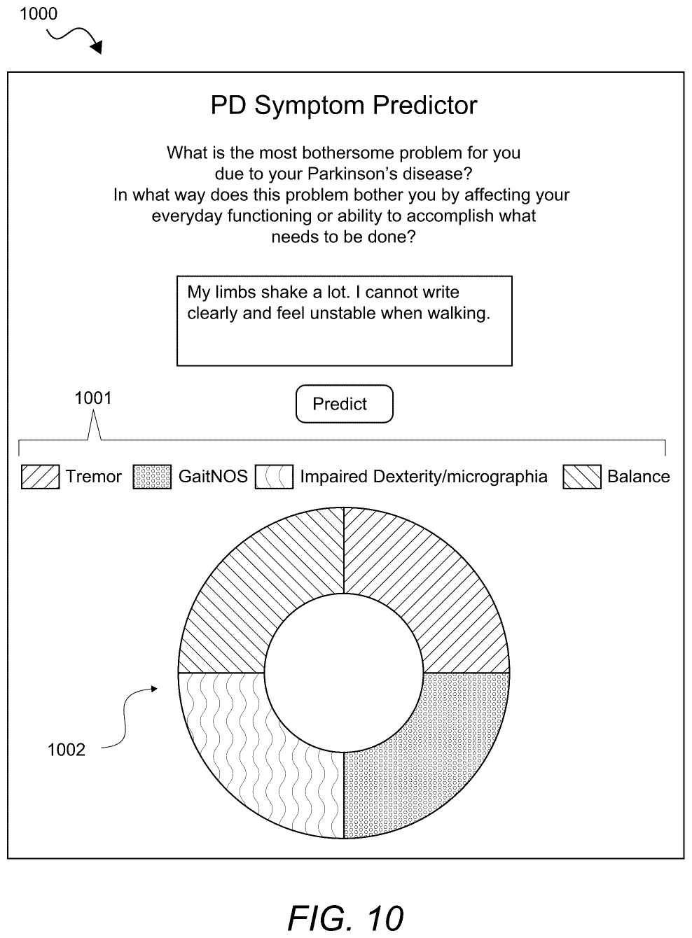

FIG. 10 is an example of the interface 1000 used by a client on the client computing device 120 to enter their plain-text input. In the example of FIG. 10, the interface 1000 is for patients suffering from Parkinson's disease.

It should be noted that the example of the interface 1000 accepts typed text inputs. However, it is contemplated that audio and/or video inputs can also be used. In embodiments where audio and/or video inputs are used, the client computing device 120 includes a microphone or a camera capable of capturing the client speaking the input. The audio and/or video data is transmitted to the computing device 110, which obtains text from the speech captured. This text is then used as discussed herein.

In another example the response to the prompt would be related to dystonia and having trouble falling asleep. Verbatim would be “dystonia and trouble falling asleep (I am exhausted because I have trouble sleeping at night, and also lack of sleep gives me migraines during the day).” The part in parenthesis would be the answers to the questions at step 901.

At step 902, the responses are concatenated into the verbatim form.

At step 903, the verbatim is fed into the inference model that is described above, as the input.

At step 904, the computing device 110 outputs the classification of the verbatim into symptoms and domains. In this example, the computing device 110 returns a result that causes the display of interface 1000 to show one or more classifications based on the plain-text input. In the example of FIG. 10, the symptom classifications 1001 (in this example, tremor, gait NOS, impaired dexterity/micrographia and balance) are listed and then graphically shown via graph 1002. The graph can also show the probabilities of the words. The classifications can also be output via audio.

Continuing with the dystonia example, the output could be:

[(‘cramping’, 0.88),
(‘dyskinesia’, 0.72),
(‘rigidity’, 0.72),
(‘tightness', 0.70),
(‘contraction’, 0.65)]

Following the display of the classifications, the computing device 110 can also use the patient's input to further refine and improve the system. Thus, at step 905, the computing device 110 takes the top synonyms returned at step 904 (for example, the top 5 synonyms) and creates a rule by adding the synonyms to the linguistic dictionary.

The verbatims are then labeled at step 906 by using the rules in the linguistic dictionary. The labels for the dystonia example could be as follows:

Labels: Domain: Other Motor, Symptom: Dystonia; Domain: Fatigue, Symptom: Physical Fatigue; Domain: Sleep, Symptom: Sleep Onset Insomnia; Domain: Pain, Symptom: Headache.

At step 907, the example verbatims received by the input can be used to train the model to detect other verbatims received in the future, that mention similar problems. For example, the verbatim “headaches and cramping (I have trouble walking) would get classified by the inference model into “dystonia” and “headaches.”

As will be appreciated by the astute reader, the systems and methods of the inventive subject matter enable a computer system to understand plain-text language and translated it to clinical verbiage, and grow and learn such that the patient interactions with the system are increasingly easy and natural for patients and accurate for their care and treatment.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A non-transitory computer readable storage medium storing instructions that, when executed by a processor, cause the processor to:
receive a plurality of verbatims, wherein each of the plurality of verbatims comprises a data item including a concatenated report of a problem and a consequence of the problem;
obtain a curated symptom definition table;
generate a linguistic dictionary based on a known sentence structure and the curated symptom definition table;
generate an additional plurality of verbatims from the received plurality of verbatims;
validate the additional plurality of verbatims;
train a model using the validated verbatim set; and
utilize the trained model to predict one or more of at least one symptom or at least one domain based on a raw-text input query.

2. The non-transitory computer-readable storage medium of claim 1, further comprising instruction to generate the linguistic dictionary by causing the processor to:
extract parts of speech received by the computing device;
train a model for synonym detection based on clinical trial and pubmed data;
perform UMLS-controlled identifier extraction to obtain a plurality of words and phrases associated with a specific symptom; and
extract at least one verbatim based on the plurality of words and phrases.

3. The non-transitory computer-readable storage medium of claim 1, further comprising wherein the symptom definition table comprises a plurality of symptoms and for each symptom, a domain to which the symptom belongs, at least one symptom inclusion, at least one symptom exclusion, and at least one sample phrase associated with the symptom.

4. The non-transitory computer-readable storage medium of claim 1, further comprising instructions that cause the processor to annotate the verbatims, and wherein each of the annotated verbatims comprises a domain to which the symptom belongs, a symptom name, a serial number, and at least one term associated with the symptom.

5. The non-transitory computer-readable storage medium of claim 4, further comprising instructions that further cause the processor to annotate each of the verbatims by generating rules for each symptom based on one or more of: a symptom inclusion and exclusion criteria, an obtained annotation and term or phrase, at least one closely-related term derived via algorithm, and ICD-10 codes.
